(19) United States
(12) Patent Application Publication
(10) Pub. No.: US 2025/0387469 A1
(43) Pub. Date: Dec. 25, 2025

(54) Use of the ultrafiltration for producing purified allergenic extracts

(71) Applicant: Anallergo S.p.A., Scarperia e San Piero FI (IT)

(72) Inventors: Neri ORSI BATTAGLINI, Scarperia e San Piero FI (IT); Alessandro BALLINI, Scarperia e San Piero FI (IT)

(21) Appl. No.: 18/861,300

(22) PCT Filed: May 31, 2023
(86) PCT No.: PCT/IB2023/055579

§ 371 Date: Oct. 29, 2024

(30) Foreign Application Priority Data
May 31, 2022 (IT) 102022000011459

(51) Int. Cl. A61K 39/35; B01D 61/14; B01D 61/58

(52) U.S. Cl. CPC A61K 39/35; B01D 61/145; B01D 61/147; B01D 61/58; B01D 2257/91; B01D 2311/14; B01D 2311/22; B01D 2325/02831; B01D 2325/02833

(57) ABSTRACT

Process for producing a finished product containing or consisting of a purified allergenic extract, the process comprising the steps of: (A) providing a crude allergenic extract, the crude allergenic extract being obtained by isolation from a biological sample; (B) optionally, cleaning the crude extract, to obtain a clean crude extract, the cleaning step comprising the sub-steps of (B1) filtering the crude extract to remove the microbial load native to the biological sample; (C) optionally, purifying the crude extract or the clean crude extract, to obtain a purified extract, the purification step comprising the sub-steps of (C1) filtering the crude extract or the clean crude extract to remove impurities having a molecular weight ≤5,000 Da; the process being characterized in that it comprises a viral ultrafiltration step functional to obtain a finished product substantially free of virus and/or mycoplasma, which contains or consists of a purified extract of the allergen.

The patent application also concerns products for medical use or for diagnostic use, comprising one or more allergen extracts substantially free of viruses and/or mycoplasmas native to the starting biological sample or from any external contamination.

### FIELD OF THE INVENTION

The present invention is placed in the technical field of the production processes of products containing extracts of allergens useful for therapeutic use, for specific immunotherapy in the treatment of patients suffering from allergies or allergic manifestations; for diagnostic use, in the detection of allergies.

### State of the Art

Specific immunotherapy (ITS) is the first choice therapy for the treatment of patients suffering from respiratory allergies or clinical manifestations of an allergic type. In addition to relieving symptoms, ITS fights the causes of the allergy.

The WHO (World Health Organization) recognizes it as the only treatment that can “lead to the healing of the allergy and change the quality of life of the patient”.

Desensitization is based on the principle of administering to the patient small and progressively increasing amounts of the allergens that cause the allergic reaction.

There are currently 2 forms of desensitizing therapy: the sublingual form, in which the allergens are left for a few minutes under the tongue. The injectable form, in which the allergens are injected by the doctor at increasing doses, until the maximum dose is reached.

The allergens causing the allergic reaction are generally extracted from samples or biological substrates of animal (mites; hymenoptera venom; pet epithelial derivatives), microbiological (mould/yeast cultures) origin or plant origin (pollen from herbs and plants). The nature of the starting material makes the allergen extracts particularly complex from a qualitative point of view, since they include both the molecules of the reference allergen and the molecules belonging to the microbiomes associated with the starting material. To give an example, the mite cultures, from which the antigens responsible for the respiratory allergies to such arthropods are extracted, contain immunoactive bacterial endotoxins, such as lipopolysaccharides (LPS), whose presence is systematically controlled during the pharmaceutical production step in order to obtain the certifications required for marketing authorisation.

Over the past decade, viral metagenomics and the development of new technologies at its service have revealed an extraordinary diversity and ubiquity of viruses in the biosphere. It was then started to investigate whether the biological materials used for the extraction of allergens could also be a source of immunogenic viral components.

The presence of viruses in the allergen extracts, in fact, could have an impact not only in the production steps of the pharmaceutical product (for example, by impacting the multiplication efficiency of the biological sample, for the purposes of industrial extraction); but also on the immunological and inflammatory response induced in the patient, since the viral proteins are known to have antigenic and immunological properties.

Vidal-Quist et al (José Cristian Vidal-Quist, Carmen Vidal, Fernando Escolar, Bart N. Lambrecht, Stephane Rombauts, Pedro Hernández-Crespo. RNA viruses in the house dust mite Dermatophagoides pteronyssinus, detection in environmental samples and in commercial allergen extracts used for in vivo diagnosis. Allergy. 2021; 00:1-12) have demonstrated that the viral infections in the mites of the species Dermatophagoides pteronyssinus are very common, regardless of the origin of the mite (grown in industrial cultures or harvested from naturally occurring colonies). The authors found the presence of 7 different viral RNAs in D. pteronyssinus.

Similar considerations have been made for the mycoplasmas, bacteria characterized by the lack of a cell wall, a factor that makes them resistant to many antibiotic treatments (these being active precisely on the synthesis of the components forming the bacterial cell wall). Many mycoplasmas are parasitic in nature, but they can attack and invade other cells, as well. The mycoplasmas can infect humans, animals and plants, making them a potential contaminant of the biological starting materials from which the allergens are extracted. Like for the viruses, their presence can also be responsible for the difficulties in the production step, slowing down and nullifying, for example, the production operations of the allergenic extracts at an industrial level (https://www.citeqbiologies.com/mycoplasma-and-house-dust-mite-extracts/).

### Problems of the Background Art

The “Viral Safety” chapter of the European Pharmacopoeia (European Pharmacopoeia, 5.1.7) provides the viral safety requirements for the medicinal products that envisage the use of materials of human or animal origin.

The use of viral inactivation steps has been used, in the state of the art, in drugs that directly use material of biological origin (e.g. blood derivatives, etc.).

To date, the risk of viral and/or mycoplasmic infection in products obtained by extraction containing allergenic proteins from starting pet material has always been considered low or non-existent.

For this reason, there is no evidence in the state of the art of the implementation of procedures suitable for abating the viral and/or mycoplasmic load in products intended for specific immunotherapy (ITS).

The authorities responsible for authorising the marketing of this type of product do not require in fact any certification attesting the content in viruses and/or mycoplasmas of the allergenic extracts.

Nevertheless, the harvesting and/or breeding processes for the preparation of the raw materials, the protein extraction process, the filtration and purification processes, are carried out with systems open to potential contact with the personnel and therefore potentially exposed to the viruses and bacteria.

Viral filtration, which removes the viruses based on a dimensional exclusion mechanism, is widely used in the purification systems of biotherapeutics derived from mammalian cell cultures. Viral filtration is generally carried out through the use of filters having mesh sizes in the order of nanometers, and in this sense called nanofilters or, sometimes, viral ultrafilters. Consequently, viral filtration is referred to as nanofiltration or sometimes as viral ultrafiltration.

However, viral filters are not easy to implement in sample purification systems, as they are easily subject to packing/clogging (also known as fouling), responsible for a decrease in the filtrate flow and, consequently, for a reduction in the virus retention.

For the typical dimensional characteristics of the viral ultrafiltration systems, in relation to surface of the filter in cm² and material of the filter, the elements that can most frequently be responsible for clogging of the system are components having molecular weight >150 kDa or protein aggregates.

The difference in terms of molecular weight between the proteins of the allergens of interest (1-150 kDa), the viruses (150-7200 kDa) and the mycoplasmas (0.2-0.3 μm) is not, in this sense, such as to guarantee an easy removal of the virus by dimensional exclusion, without risking compromising the yield of the extract in terms of protein content.

It should also be noted that the allergenic proteins are unstable in aqueous solution (both at room temperature and under refrigerated conditions), unless it is resorted to adding stabilising excipients or techniques that improve their stability (such as freeze-drying); therefore, the application of a viral ultrafiltration step may be a reason of reduction or loss of the protein content by degradation of the molecules of interest.

In addition to degradation, the protein content can also be influenced by phenomena of absorption of individual proteins of interest on the filter membranes.

In addition to the aforementioned problems, it should also be taken into considering that the implementation of the viral ultrafiltration in a production system normally requires an adaptation of the working conditions to the production process being implemented, not only according to the species contained in the sample to be filtered, but also among different batches of the same product. The viral ultrafiltration process, in other words, is subject to some variability and is hardly standardizable.

### SUMMARY OF THE INVENTION

In order to reduce the risk of contaminations by microorganisms, viruses and/or mycoplasmas, the Applicant has developed a process for producing a finished product containing or consisting of a purified allergenic extract, substantially free of viruses and/or mycoplasmas; in particular, the process object of the invention sees implemented a viral ultrafiltration step, dedicated to viral and/or mycoplasmic removal.

Object of the present invention is therefore a process for producing a finished product containing or consisting of a purified allergenic extract, the process comprising the steps of:
A) providing a crude allergenic extract, in which the allergen is of natural origin and can be obtained from a biological sample;
B) optionally, cleaning the crude extract, to obtain a clean crude extract, the step of cleaning comprising at least the sub-step of
   B1) filtering the crude extract to remove the microbial load of the biological sample;
C) optionally, purifying the crude extract or the clean crude extract, to obtain a purified extract, the step of purifying comprising at least the sub-step of
   C1) filtering the crude extract or the clean crude extract to remove impurities having a molecular weight ≤5,000 Da,
the process being characterized in that it comprises one or more viral ultrafiltration steps, said ultrafiltration being functional to obtain a finished product substantially free of viruses and/or mycoplasmas, containing or consisting of the purified allergenic extract.

A further object of the present invention is a composition substantially free of viruses and/or mycoplasmas, comprising one or more allergen extracts, which can be obtained with the process described above. The composition is preferably intended for medical use, for example in the specific immunotherapy of allergic subjects; or for diagnostic use, for the detection of allergies.

### Advantages of the Invention

The development of the process object of the invention has led to overcoming the problems of the known art, with the achievement of the following advantages and objectives:
obtaining finished products or allergenic extracts substantially free of microorganisms, viruses and/or mycoplasmas native to the biological starting material or coming from external contaminations: the products thus characterised are safer for the end user, since they are free of antigenic substances that could interfere with the immunospecific therapy.
implementation of a viral ultrafiltration step which, in addition to being functional to the reduction of the content of viruses and/or mycoplasmas in finished products comprising or consisting of allergenic extracts, was also found to have an impact on the control of the content of bacterial endotoxins (see Example 2).
implementation of an efficient viral ultrafiltration step, which does not suffer from fouling problems. This object was achieved specifically through the use of a filter assembly comprising a pre-filter, upstream of the main filter. The implementation of this ultrafiltration step has been further improved by the selection of appropriate dimensions of the pre-filter and of the subsequent filter, which would allow, at sustainable pressures for the system (0.5-5 bar), to obtain a rapid ultrafiltration, to retain specifically viruses and/or mycoplasmas, letting instead the allergenic proteins pass.
implementation of a viral ultrafiltration step that does not impact the yield of the product, in terms of the protein content of the allergens of interest; the Applicant, in particular, has experimented that the pore sizes of the filter do not negatively influence the protein and allergenic content of the active substances tested.
definition of a working pressure, which would allow the viral ultrafiltration step to be carried out between 2 and 24 hours.
obtaining a production process that, overall, allows to produce purified allergen extracts, substantially free of viruses and/or mycoplasmas, within 42 hours from the provision of a crude allergenic extract.
standardisation of the viral ultrafiltration process, as well as of the entire production process, regardless of the protein species contained in the extract and of the different batches. The process can, in fact, be used for the production of products containing or consisting of allergen extracts of different nature.

### DESCRIPTION OF THE DRAWINGS

FIG. 1: Schematic representation of the viral ultrafiltration system.

FIG. 2 (A, B, C): Flow diagram of the production process in accordance with the invention, and specifically with the applicable production process for obtaining a purified allergenic extract from mites.

### DETAILED DESCRIPTION OF THE INVENTION

### Definitions

“Allergen” means an antigen or a set of antigens that elicits an unwanted immune hypersensitivity reaction (or allergic reaction). What is “allergenic” is related to the allergen so defined. This definition applies to the whole following descriptions, regardless of whether reference is made to the allergen in the singular form or to the allergens in the plural form.

“Allergen of natural origin” means an allergen that is naturally occurring and that has not been manipulated or modified by genetic engineering or molecular biology techniques.

“Raw extract” (or crude extract) of allergen means the extract obtained by isolating and separating the allergen from the starting material (or biological sample) in which it was originally contained (i.e. obtained by applying an extraction process directly to the allergen source). In other words, a crude allergen extract contains the proteins as such isolated and separated from the source material, regardless of their degree of purity. Th extracts that possibly can be obtained by subsequent manipulation of the raw extract (or crude extract) are to be considered purified extracts (and no longer raw or crude extracts).

By “purified, substantially free of viruses and/or mycoplasmas” is meant a product that has undergone, in addition to the purification processes adapted to eliminate molecules not belonging to the allergen and to the processes of abating the microbial load of the starting biological sample or coming from external contaminations, also the specific processes of removal of viruses and/or mycoplasmas. The viruses and the mycoplasmas that are removed can be both those native to the starting biological sample and those coming from any external contaminations. The purified product, substantially free of viruses and/or mycoplasmas, is the one that complies, in terms of quantity/quality, with the industry regulations (ICH Harmonized Tripartite Guideline Q5A (R1), 1999: Viral Safety evaluation of biotechnology products derived from cell lines of human or animal origin. https://www.ema.europa.eu/en/ich-q5a-r1-quality-biottechnological-products-viral-safety-evaluation-biotechnology-products-derived: EMEA/CPMP/BWP/268/95/3AB8A, 1996: Note for guidance on virus validation studies: The design, contribution and interpretation of studies validating the inactivation and removal of viruses).

“Finished product” means a product suitable for distribution and sale endowed with its own biological activity. The finished product may, as such, constitute a product usable for therapeutic or diagnostic purposes; or it may constitute a raw material, suitable for being further manipulated or mixed with other ingredients for the preparation of products that can be used for therapeutic or diagnostic purposes.

By “protein dosage” is meant the quantitative content of proteins in a sample; within the purposes of the invention, the protein dosage makes it possible to determine, with a relative specificity, the allergen content present in the reference extract.

By “Lowry method or assay” is meant a method for quantifying the protein dosage of a sample, known in the art (LOWRY O H, ROSEBROUGH N J, FARR A L, RANDALL R J. Protein measurement with the Folin phenol reagent. J Biol Chem. 1951 November; 193 (1): 265-75. PMID: 14907713). The Lowry assay is based on a two-step procedure: (1) the biuret reaction: by adding a copper solution to the protein solution in a basic environment, a purple-violet colouring with a maximum absorption at 540 nm is obtained. (2) After the addition of the Folin's reagent (phosphomolybdic-phosphotungstic reagent) which reacts with the tyrosines and the tryptophanes of the proteins, it assumes a blue colour (with a maximum of 750 nm) given by the formation of tungsten blue and molybdenum blue thanks to the reduction operated by the copper-protein complex.

“Native to the biological sample” means a material (whether microbial, bacterial, viral, mycoplasmatic) that originates from or is contained in the starting biological sample from which the allergen is extracted.

By “filtrate” is meant the fraction of product subjected to filtration that crosses the membrane of a filter system, i.e. that is not retained by the membrane. The “filtrate” may also be referred to by the name “permeate”.

By “retentate” is meant the fraction of product subjected to filtration that does not cross the membrane of a filter system, i.e. that is retained by the membrane.

Hereinafter, the invention is described with greater detail. With reference to the process, the steps and the sub-steps constituting the same will be described.

### A—Providing a Crude Allergen Extract.

As already mentioned above, the allergen contained in the crude extract is a natural allergen and obtainable from a biological sample.

Preferably, the allergen contained in the crude extract for the purposes of the invention is an allergen of animal origin, obtainable from a biological sample containing or consisting of material classified as belonging to the Animalia kingdom.

Still preferably, the allergen contained in the crude extract for the purposes of the invention is a hymenoptera or mite or mammalian (preferably dog or cat) allergen.

According to a preferred embodiment, the isolation of the crude allergen extract from the biological sample can take place separately and in a place different from where the process of the invention is carried out; or it can constitute an integrating step of the production process.

In case the isolation of the crude extract from the biological sample constitutes an integrating step of the production process object of the invention, the step of providing the crude allergen extract (A) preferably comprises at least or consists of a sub-step of isolating the allergen from a biological sample (or starting material) (A1).

Isolation, for the purposes of the invention, means the set of techniques used for the separation of a crude allergen extract from the starting biological sample; the isolation, as the case may be, may therefore comprise solvent extraction and/or crude extract separation techniques.

The biological sample (or starting material) can be selected from:
mites, preferably in the form of dried and frozen mite cultures; the cultures may include different mite components (e.g. faecal pellets, mite bodies, parts of mites, eggs).
mammalian material, preferably selected from epidermal material, mammalian hair or dandruff, preferably selected from mammalian hair and dandruff.
hymenoptera, preferably selected from the venom bag from the whole insect, the unprocessed venom in liquid form, the processed and unprocessed venom in lyophilic form.

According to a first preferred embodiment, the biological sample (or starting material) consists of mites. Preferably the mite species chosen for the purposes of the invention are Dermatophagoides farinae, Dermatophagoides pteronyssinus or Euroglyphus maynei.

In the case of mites, the crude allergen extract can be obtained with methods known to the person skilled in the art (Prester, Ljerka, Kovačić, Jelena and Macan, Jelena. “Comparison of buffers for extraction of mite allergen der p 1 from dust” Archives of Industrial Hygiene and Toxicology, vol. 63, no. 3, 2012, pp. 293-300). For example, a mite culture may be mixed with an aqueous solvent of an ionic nature. The crude extract is isolated from the extraction medium by traditional separation techniques (centrifugation, filtration).

According to a second preferred embodiment, the mammalian biological sample (or starting material) consists of samples of material coming from a dog (Canis lupus) or from a cat (Felis catus), preferably from a cat.

In the case of a dog or a cat, the crude extract of epidermal material, hair or dandruff of the pet can be obtained using methods known to the person skilled in the art. Generally, the material is collected in animal storage facilities, under the supervision of a veterinarian (Fernández-Caldas E, Cases B, El-Qutob D, Cantillo J F. Mammalian raw materials used to produce allergen extracts. Ann Allergy Asthma Immunol. 2017 July; 119 (1): 1-8).

According to a third preferred embodiment, the biological sample (or starting material) consists of hymenoptera venom. Preferably, the species of hymenoptera chosen for the purposes of the invention are Apis mellifera, Vespa Crabro, Vespa velutina, Vespa mandarinia, Vespula germanica, Vespula vulgaris, Polistes dominula, Polistes exclamans, Bombus terrestris, Bombus agrorum, Bombus medics, Dolichovespula, Vespula alascensis, Vespula maculifrons, Vespula flavopilosa, Vespula pensylvanica and Vespula Squamosa; still preferably selected from among Apis mellifera, Vespa Crabro, Vespula germanica, Polistes exclamans, Bombus terrestris, Bombus agrorum, Bombus medics, Polybia paulista.

In the case of hymenoptera venom, the crude extract is obtainable with methodologies known to the person skilled in the art (Greg Plunkett, PhD; Robert S. Jacobson, M S; David B. K. Golden, M D. Hymenoptera venoms used to produce allergen extracts. Ann Allergy Asthma Immunol 118 (2017) 649-654). For example, the venom bag is recovered from the insect (live, after capture or from breeding) by dissection and the venom recovered with appropriate recovery technique. The venom can be extracted from the bag by breaking it in an appropriate buffer, to allow the release of the venom. The residues of the bag are removed by centrifugation and filtration. The venom thus obtained constitutes the crude allergen extract from hymenoptera for the purposes of the invention. The venom in liquid form, not subjected to processing, also constitutes a crude extract of hymenoptera allergens; that said, it is less preferred, for subsequent processings, due to its high instability, having a predominantly enzymatic composition. A further example of crude extract of hymenoptera allergens is the liquid venom extracted from the bag and processed with operations such as dissolution, filtration and/or freeze-drying, thanks to which the extracted venom is treated in such a way as to ensure stability over time.

Note that regardless of the source of the allergen, the crude extract has a total microbial load ≤1×10¹² CFU/g.

In the most preferred embodiment of the invention, the starting crude allergenic extract (provided in step (A)) is selected from the group consisting of
crude allergenic extracts of mites, preferably of Dermatophagoides farinae, or Dermatophagoides pteronyssinus or Euroglyphus maynei; or
crude allergenic extracts of dandruff and/or hairs of Felis catus; or
crude allergenic extracts of hymenoptera venom, preferably of Apis mellifera, or Vespa Crabro, or Vespa velutina, or Vespa mandarinia, or Vespula germanica, or Vespula vulgaris, or Polistes dominula, or Polistes exclamans, or Bombus terrestris, or Bombus agrorum, or Bombus medics, or Dolichovespula, or Vespula alascensis, or Vespula maculifrons, or Vespula flavopilosa, or Vespula pensylvanica or Vespula Squamosa; still preferably selected from allergenic extracts of Apis mellifera, Vespa Crabro, Vespula germanica, Polistes exclamans, Bombus terrestris, Bombus agrorum, Bombus medics, Polybia paulista.

In accordance with this most preferred embodiment, the step of providing the crude allergen extract is carried out in grade C and/or D environments according to ISO 14644-1.

Note that, when the biological sample consists of a mite culture, for the purpose of obtaining the crude extract the sample is preferably mixed with a phosphate buffer as extraction solvent. Preferably the phosphate buffer has a pH ranging from 7.5 to 8.5, preferably equal to about 8.0.

The mixture is stirred in such a way as to favour the mixing of the steps. At the end of stirring, the pH of the mixture can be adjusted with alkaline agents, acids or other buffers, so that the pH of the mixture ranges from 7.5 to 8.5, preferably equal to about 8.0.

The mixture is then allowed to stand, for a time preferably ranging from 12 to 72 hours for the extraction to take place. For the purpose of extracting the mite allergens, a cold extraction is preferred, carried out at a temperature ranging from 2° to 8° C.

For the purpose of isolating the extract, common separation techniques may be used. Preferably, the mixture is subjected to centrifugation, recovering the supernatant (which constitutes the crude extract of allergens of mites) and removing the precipitate.

Note that when the starting material consists of hair or dandruff of Felis catus, the extraction of the crude extract takes place in the same manners as described above for dust mites.

### B—Cleaning the Crude Extract, to Obtain a Clean Crude Extract.

According to a preferred embodiment, the process object of the present invention comprises a step of cleaning the crude allergen extract (B). The step of cleaning the crude allergen extract (B) comprises at least or consists of the sub-step of filtering the crude extract to remove the microbial load of the biological sample (B1).

The cleaning step (B), in this sense, is not necessary when the starting crude extract is already sufficiently free of bacterial contaminants; preferably, the cleaning step (B) is avoidable as much as the starting crude allergen extract is characterized by a microbial load <100,000 CFU/ml, preferably ≤50,000 CFU/ml.

According to a preferred embodiment, the sub-step of microbial filtration of the crude extract (B1) comprises or consists of the sub-steps of:
performing a first dimensional filtration, using a first filter with a porosity of dimensions ranging from 8 μm to 20 μm, preferably ranging from 8 μm to 10 μm, preferably equal to 8 μm;
performing a second dimensional filtration, after the first, using a second filter with a porosity of dimensions ranging from 0.1 μm to 0.2 μm, preferably equal to 0.2 μm.

Note that the retentate obtained from the sub-step of filtering the crude extract constitutes the microbial load of the biological sample and, as such, is discarded for the purposes of the subsequent steps of the method in accordance with the invention; the permeate (or filtrate) obtained from the sub-step of filtering the crude extract constitutes the clean crude extract or a precursor of the clean crude extract.

Preferably, the sub-step of filtering the crude extract (B1) is a traditional filtration or “dead end” filtration, i.e. carried out by applying a pressure perpendicularly to the membrane from the supply side of the sample to be subjected to filtration.

Preferably, the first filter used in the sub-step of microbial filtration of the crude extract (B1) is a polypropylene (PP) filter.

Preferably, the second filter comprises a pre-filter with a porosity of dimensions ranging from 0.45 μm to 0.65 μm, preferably from 0.45 μm to 0.50 μm, preferably equal to 0.45 μm.

Preferably, the pre-filter is structurally coupled to the filter, so as to define a filter assembly in a solution of continuity. The presence of the pre-filter allows to avoid clogging of the second filter, contributing to the definition of a production process that ends in or within 42 hours from the start of the step of providing the crude extract (A), preferably at a pressure ranging from 0.5 bar to 5 bar, preferably from 1.5 bar to 4 bar, preferably ranging from 1.8 bar and 3 bar, preferably equal to about 2 bar.

Preferably, both the second filter and the relative pre-filter, where present, are made of polyethersulfone (PES).

Note that the clean crude extract is preferably characterized by a microbial load <100,000 CFU/ml, preferably ≤50,000 CFU/ml.

Note that the clean crude extract is preferably characterized by a protein dosage (Lowry assay) ranging from 150% to 450% by weight of the protein dosage of the final purified extract (the dosage of the final purified extract corresponds to 100%).

Preferably, the protein dosage of the clean crude extract of mite allergens, the mites preferably being Dermatophagoides farinae or Dermatophagoides pteronyssinus or Euroglyphus maynei, ranges from about 3 mg/mL to 9 mg/mL.

Preferably, the protein dosage of the clean crude extract of pet animal allergens, the pet animals being preferably selected from Canis lupus and Felis catus, ranges from about 0.4 mg/mL to 9 mg/mL.

Preferably, the protein dosage of the clean crude extract of hymenoptera venom allergens, the hymenoptera being preferably selected from Apis mellifera, Vespa Crabro, Vespa velutina, Vespa mandarinia, Vespula germanica, Vespula vulgaris, Polistes dominula, Polistes exclamans, Bombus terrestris, Bombus agrorum, Bombus medics, Dolichovespula, Vespula alascensis, Vespula maculifrons, Vespula flavopilosa, Vespula pensylvanica, Vespula Squamosa and Polybia paulista ranges from about 0.3 mg/mL to 9 mg/mL.

It should be noted that, preferably, said clean crude extract of hymenoptera venom allergens is in dry-frozen form; the indicated protein dosage therefore refers to the dosage of the extract before freeze-drying or, after freeze-drying, when the lyophilizate is reconstituted in 1 mg/mL of saline solution.

Particularly preferred embodiments of hymenoptera include Apis mellifera, Vespa Crabro, Vespula germanica, Polistes exclamans, Bombus terrestris, Bombus agrorum, Bombus medics.

According to a preferred embodiment, the step of cleaning the crude extract, in order to lower the microbial content of the extract, is carried out in grade D environments according to ISO 14644-1.

### C—Purifying the Clean Crude Extract to Obtain a Purified Extract.

According to a preferred embodiment, the process object of the present invention comprises a step of purifying the crude extract (in the case where a step of cleaning the crude allergen extract (B) has not been carried out beforehand) or a step of purifying the clean crude extract (in the case where, instead, the crude extract had to undergo a cleaning step (B) beforehand).

The step of purifying the crude extract or the clean crude extract (C) comprises at least or consists of the sub-step of filtering the crude extract or the clean crude extract to remove impurities having a molecular weight ≤5,000 Da (C1) (alternatively said “sub-step of removing impurities from the clean crude extract”).

These impurities are molecules that do not belong to the allergen and contain salts and/or proteins native to the starting biological sample or introduced in the previous steps of the process (think, for example, of the solvent solutions that can be used during the extraction step). It is important that these impurities are removed from the sample to regulate the isotonicity of the final extract, as well as to prevent the specific immunotherapy product from being able to give sensitization reactions due to other protein species.

In this sense, therefore, the step of purifying the crude extract or the clean crude extract is not necessary when the isotonicity of the sample is already suitable for the preparation of a finished product. By way of example, in the production of finished products comprising or consisting of hymenoptera venom allergens, it is not always necessary to carry out a step of purifying the crude extract, since there are mechanical methods of extracting the venom that do not require the use of solvents that may impact on the isotonicity of the crude extract.

Preferably, the sub-step of removing impurities from the crude extract or from the clean crude extract (C1) is carried out by diafiltration, preferably in tangential mode (tangential diafiltration), preferably using a filter with dimensional cut-off of 5,000 Da.

The advantage in the use of diafiltration, preferably in tangential mode (tangential diafiltration) at this step of the production process consists in the possibility of removing molecules having a significantly lower molecular weight than the allergenic proteins of interest which, precisely for this reason, could not be eliminated in any previous filtration steps (such as, for example, in the cleaning step (B)). During diafiltration, the impurities pass through the membrane and are not retained (constituting the “permeate” or “filtrate”). The remaining part of the diafiltration extract, retained by the membrane and containing proteins and molecules with a molecular weight greater than 5,000 Daltons, constitutes the “retentate”.

Still preferably, the sub-step of removing the impurities from the clean crude extract (C1) comprises the following steps, carried out using the same filter (or membrane):
a first passage of the clean crude extract through the membrane with cut-off of 5,000 Da, to obtain a concentrated form of the clean crude extract (retentate);
at least four further passages through the same membrane with cut-off of 5,000 Da, washing the retentate obtained from each of the previous passages with saline solution.

According to a preferred embodiment, the impurity-deprived extract in accordance with step (C1) can be further filtered, using a filter with a porosity of dimensions ranging from 0.1 μm to 0.2 μm, preferably equal to 0.1 μm.

The extract obtained from the sub-step of removing the impurities from the clean crude extract (C1), either it being comprehensive or without further downstream filtration (membrane 0.1-0.2 μm), constitutes a purified extract.

Note that such purified extract is preferably characterized by a microbial load <100,000 CFU/ml, preferably ≤50,000 CFU/ml.

Note that the purified extract is preferably characterized by a protein dosage ranging from 100% to 300% by weight of the protein dosage of the final purified extract (the dosage of the final purified extract corresponds to 100%).

Preferably, the protein dosage of the purified extract of mite allergens, the mites preferably being Dermatophagoides farinae or Dermatophagoides pteronyssinus or Euroglyphus maynei, ranges from about 1.9 mg/mL to 6.0 mg/mL.

Preferably, the protein dosage of the purified allergen extract from pet animals, the pet animals preferably being Canis lupus or Felis catus, ranges from about 0.25 mg/mL to 6.0 mg/mL.

Preferably, the protein dosage of the purified extract of hymenoptera venom allergens, the hymenoptera being preferably selected from Apis mellifera, Vespa Crabro, Vespa velutina, Vespa mandarinia, Vespula germanica, Vespula vulgaris, Polistes dominula, Polistes exclamans, Bombus terrestris, Bombus agrorum, Bombus medics, Dolichovespula, Vespula alascensis, Vespula maculifrons, Vespula flavopilosa, Vespula pensylvanica and Vespula Squamosa, ranges from about 1.0 mg/mL to 3.0 mg/mL. Still preferably, the hymenoptera are selected from Apis mellifera, Vespa Crabro, Vespula germanica, Polistes exclamans, Bombus terrestris, Bombus agrorum, Bombus medics, Polybia paulista ranges from about 0.2 mg/mL to 6.0 mg/mL.

According to a preferred embodiment, the step of purifying the clean crude extract is carried out in grade D environments according to ISO 14644-1.

### D, E—Providing the Extract for Packaging and Packaging.

According to a preferred embodiment, the production process of the invention comprises other steps, subsequent to the purification step (C), aimed at packaging the purified extract (D, E).

Preferably, the purified allergen extract is brought back to the desired titre (100%), in terms of protein dosage, by adding saline solution to the purified allergen extract (D).

The extract obtained from this step of back titration constitutes the purified and titrated allergenic extract.

Preferably, the protein dosage of the purified and titrated extract of mite allergens, the mites preferably being Dermatophagoides farinae or Dermatophagoides pteronyssinus or Euroglyphus maynei, ranges from about 1.9 mg/mL to 2.4 mg/mL.

Preferably, the protein dosage of the purified and titrated extract of pet animal allergens, the pet animals preferably being Canis lupus or Felis catus, ranges from about 0.25 mg/mL to 2.0 mg/mL.

Preferably, the protein dosage of the purified and titrated extract of hymenoptera venom allergens, the hymenoptera being preferably selected from Apis mellifera, Vespa Crabro, Vespa velutina, Vespa mandarinia, Vespula germanica, Vespula vulgaris, Polistes dominula, Polistes exclamans, Bombus terrestris, Bombus agrorum, Bombus medics, Dolichovespula, Vespula alascensis, Vespula maculifrons, Vespula flavopilosa, Vespula pensylvanica and Vespula Squamosa, and still preferably selected from Apis mellifera, Vespa Crabro, Vespula germanica, Polistes exclamans, Bombus terrestris, Bombus agrorum, Bombus medics, Polybia paulista ranges from about 0.2 mg/mL and to 2.0 mg/mL.

Note that preferably the step of back titration (D) is carried out in a grade C room according to ISO 14644-1.

According to a preferred embodiment, before packaging, the purified and titrated allergenic extract is subjected to a final cleaning step, to remove any additional microbial load, accumulated in the previous processing steps (E). In other words, such further microbial load is not to be considered native to the starting biological sample.

According to a preferred embodiment, the step of microbial filtration of the purified and titrated extract (E) comprises or consists of the sub-steps of:
performing a dimensional filtration by means of a filter with a porosity of dimensions ranging from 0.1 μm to 0.2 μm, preferably equal to 0.1 μm.

The extract obtained from the step of microbial filtration of the purified and titrated extract (E) constitutes an embodiment of the finished product in accordance with the present invention.

Note that the filtrate or permeate obtained from the step of microbial filtration of the purified and titrated extract (E) contains the allergenic proteins of interest and, in this sense, may constitute the finished product in accordance with the present invention; the retentate obtained from the step of microbial filtration of the purified and titrated extract (E), on the other hand, will be discarded as it contains the microbial load non-native to the biological sample (if present).

Preferably, the step of filtering the purified and titrated extract (E) is a traditional filtration or “dead end” filtration, i.e. carried out by applying a pressure perpendicularly to the membrane from the supply side of the sample to be subjected to filtration. The force guiding the filtration is the pressure difference (differential pressure) between the inlet side and the outlet side of the filtrate.

Preferably, the filter includes a pre-filter with a porosity of dimensions ranging from 0.45 μm to 0.65 μm, preferably from 0.45 μm to 0.50 μm, preferably equal to 0.45 μm. The pre-filter is preferably structurally coupled to the filter, so as to define a filter assembly in a solution of continuity.

Preferably, both the second filter and the relative pre-filter, where present, are made of polyethersulfone (PES).

Note that the purified and titrated crude extract is preferably characterized by a microbial load <100,000 CFU/ml, preferably ≤50,000 CFU/ml.

### Viral Ultrafiltration (or Nanofiltration).

According to a preferred embodiment of the invention, the production process comprises a viral ultrafiltration step, functional to obtain a product substantially free of viruses and/or mycoplasmas, which contains or consists of a purified extract of the allergen.

The viral ultrafiltration step is preferably carried out using a filter assembly comprising a dimensional filter containing:
a filter, with a porosity of dimensions ranging from about 20 nm to 100 nm, preferably from 20 nm to 80 nm, preferably from 20 nm to 50 nm, preferably from 20 nm to 27 nm, preferably equal to 20 nm;
preferably a pre-filter, coupled at the up front (or upstream of) the filter, with a porosity of dimensions ranging from about 100 nm to 900 nm, preferably ranging from 100 nm to 600 nm; preferably ranging from 100 nm to 200 nm, preferably equal to 100 nm.

Note that, in the case of the viral ultrafiltration step, it is the retentate that is discarded, since it contains the viruses and/or mycoplasmas and, possibly, small protein fractions, in the cases of allergenic proteins with dimensions closer to 150 kDa; the permeate obtained from the viral ultrafiltration step, on the other hand, does not comprise viruses and/or mycoplasmas but constitutes, within the purposes of the invention, the purified extract of the allergen.

Preferably the filter and the pre-filter usable in the viral ultrafiltration step are polyethersulfone (PES) or polyamide (PA) filters.

Preferably, the pre-filter is structurally coupled to the filter, so as to constitute, together with the filter, a filter assembly in a solution of continuity.

Still preferably, the ultrafiltration step is carried out at a constant pressure ranging from about 0.5 to 5 bar, preferably from about 1 to 5 bar, preferably from about 1.5 to 4 bar, preferably from about 1.8 to 3 bar, preferably equal to about 2 bar.

Note that, advantageously, the ultrafiltration thus carried out can be concluded between 2 and 24 hours, preferably between 2 and 12 hours, preferably between 2 and 6 hours, preferably between 2 and 4 hours, preferably between 2 and 3 hours, preferably in 2 hours; still preferably, such ultrafiltration times are possible at a flux preferably ≥13 L·m⁻²·h⁻¹, preferably ranging from 13 L·m⁻²·h⁻¹ to 30 L·m⁻²·h⁻¹, preferably from 20 to 30 L·m⁻²·h⁻¹, preferably from 25 L·m⁻²·h⁻¹ to 30 L·m⁻²·h⁻¹.

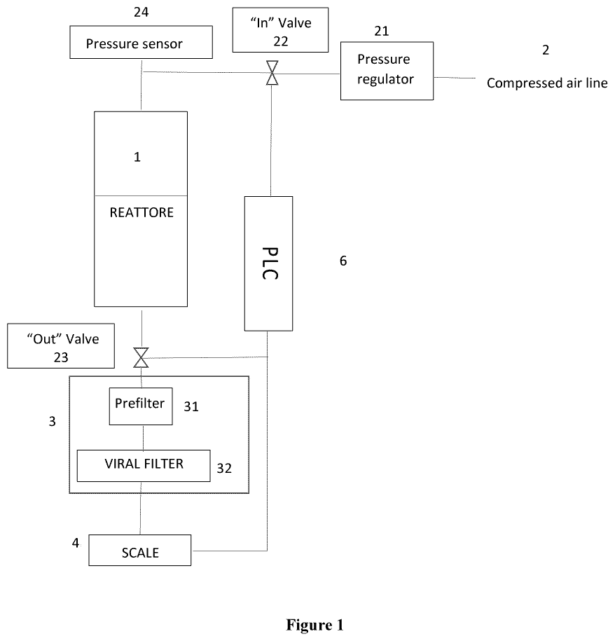

FIG. 1 schematically depicts the viral ultrafiltration system usable within the purposes of the present invention. The description of the viral ultrafiltration system is intended to describe a mode of implementation of the ultrafiltration step of a process of production of a finished product containing or consisting of an allergenic extract, but is not intended to limit the definition of the modes of exploitation of the teachings of the invention.

The system comprises a pressurizable container 1 (or “tank”), preferably in stainless steel; the container is connected, upstream, to a compressed air line 2; downstream, to a filter assembly 3 in accordance with the present invention. According to a preferred embodiment, the filter assembly comprises a filter 31 for ultrafiltration. A pre-filter 32 is coupled at the up front of the filter in accordance with the present invention.

The container 1 is preferably removably connected to the compressed air line 2, upstream, and to the filter assembly 3, downstream, so as to be able to be assembled and disassembled in the steps of washing or loading the sample. According to an alternative embodiment, the container is configured to connect to a line for feeding the sample to be subjected to viral ultrafiltration (not depicted).

The compressed air line 2 preferably comprises a pressure regulator 21, positioned upstream of the container, and an introduction valve 22 (or “in-valve”) which regulates the entry of pressurized air from the compressed air line towards the container.

The viral ultrafiltration system also comprises a discharge valve 23 (or “out-valve”), positioned downstream of the container and upstream of the filter assembly; the discharge valve regulates the flow of material exiting from the container 1 and entering the filter assembly 3.

The compressed air line 2 preferably comprises a pressure sensor 24, for reading the pressure inside the viral ultrafiltration system.

The compressed air line is connected to a separate system, which allows air to be generated under pressure (not depicted).

The “driving” force of the viral ultrafiltration system is the pressure difference (or differential pressure) between the inlet of the sample in the ultrafilter, and the outlet of the filtrate. The pressure is applied perpendicularly to the membrane of the ultrafilter, from the supply side. The guiding force is the pressure difference (differential pressure) between the inlet side and the outlet side of the filtrate.

According to a preferred embodiment, the system comprises an instrument for the volumetric or weight measurement of the filtrate 4 (in the figure, by way of example, scales), the instrument being positioned downstream of the filter assembly 3 and in communication with the filter assembly 3 by means of a discharge line. Controlling the volume or the weight of the filtrate is useful for monitoring the consistency and the accuracy of the production process.

Still preferably, the viral ultrafiltration system comprises a programmable logic controller 6 (PLC) for controlling the steps of the process. The PLC can be connected with the introduction and discharge valves (22, 23); with the instrument for the volumetric or weight measurement (4) as a function of the step of the process, or with a production management system (not depicted).

Advantageously, the process of production of the purified extract according to the present invention can be carried out in or within 42 hours.

The reduction of the amount of virus/mycoplasmas is usually expressed in terms of logarithmic reduction factor (LRF; sometimes also expressed simply as logarithmic units or “log₁₀”) and is calculated as the ratio between the viral/mycoplasmic titre of the sample before and after filtration ((ICH Harmonized Tripartite Guideline Q5A (R1), 1999: Viral Safety evaluation of biotechnology products derived from cell lines of human or animal origin. EMEA/CPMP/BWP/268/95/3AB8A, 1996: Note for guidance on virus validation studies: The design, contribution and interpretation of studies validating the inactivation and removal of viruses.).

Preferably, the viral ultrafiltration step (and thus the entire production process) reduces the amount of viruses/mycoplasmas by at least 4 log₁₀ (LRF≥4 log₁₀), preferably by at least 6 log₁₀ (LRF≥6 log₁₀), preferably by at least 8 log₁₀ (LRF≥8 log₁₀).

Still preferably, the viral ultrafiltration step (and thus the entire production process) reduces the amount of viruses/mycoplasmas of an LRF ranging from 4 to 10 (including the extremes), preferably ranging from 4 to 8 (including the extremes).

The amount of virus in a sample may for example be determined by counting the viral particles present in the sample; this count may be made by methodologies known to the person skilled in the art, such as TCID₅₀ assays, plate assays, PCR assays or electron microscopy.

The amount of mycoplasmas in a sample can for example be determined by counting the colony forming units (CFU), which can be carried out for example under a light microscope or electron microscope; or by calculating the genomic copies of the mycoplasmic DNA, by qPCR (quantitative Real-Time Polymerase Chain reaction).

Preferably, the product resulting from the viral ultrafiltration step contains a number of viral particles ≤10⁶, preferably ≤10⁴, preferably ≤10³, preferably ≤10², preferably ≤10, preferably ≤1, preferably ≤0.1, preferably ≤0.01.

By way of example, the viruses that are eliminated thanks to the implementation of the viral ultrafiltration step can be of the type Parvovirus, Retrovirus, Picornavirus.

Preferably, the product resulting from the viral ultrafiltration step has a mycoplasma content ≤50,000 CFU/mL≤10,000 CFU/mL, preferably ≤1,000 CFU/ml, preferably ≤100 CFU/ml, ≤10 CFU/ml.

By way of example, the mycoplasmas that can be eliminated thanks to the implementation of the ultrafiltration step belong to the Mollicute class (Spiroplasma, Mycoplasma and Acholeplasma).

As already mentioned, the viral ultrafiltration has also proven effective in abating the content of bacterial endotoxins: therefore, the finished products that can be obtained with the process object of the invention are not substantially free of viruses and/or mycoplasmas, but are also substantially free of bacterial pathogens, preferably of bacterial endotoxins, thanks to the implementation of the viral ultrafiltration step. Note, in any case, that—if applied—the step of cleaning the crude extract (B) contributes to abating the bacterial load of the crude extract.

Preferably, the viral ultrafiltration step is capable of abating the content of bacterial endotoxins by a value above 90% or by above 1 log₁₀, preferably by a value ranging from about 90% to 99.9% or ranging from about 1 log₁₀ to 3 log₁₀, still preferably ranging from about 90% to 99.7% or ranging from about 1 log₁₀ to 2.5 log₁₀.

Note that the content of bacterial endotoxins can be determined according to methods known in the state of the art (e.g. Turbidimetric kinetic method, Chromogenic kinetic method, Chromogenic or endpoint method, Turbidimetric endpoint method), preferably by chromogene (or chromogenic) method.

The ultrafiltration step may be carried out at one or more moments in the production process object of the invention. At least three preferred embodiments of implementation of the viral ultrafiltration step in a process for producing a finished product containing or consisting of a purified extract of an allergen are set forth below; a person skilled in the art will understand that such preferred embodiments are exemplary but not limiting the embodiments of the invention, which may also be carried out otherwise on the basis of the teachings contained in the present application.

### 1st Embodiment

According to a first preferred embodiment of the production process of the invention, the viral ultrafiltration step is carried out before the step of filtering the clean crude extract (C).

Still preferably, the viral ultrafiltration step is carried out on the crude extract of the allergen.

The viral ultrafiltration step may also be carried out after the step of cleaning the crude allergenic extract (B), and still preferably after the sub-step of filtering the crude extract to remove the microbial load of the biological sample (B1). The viral ultrafiltration step is preferably carried out on the clean crude extract.

### 2nd Embodiment

According to a second preferred embodiment of the production process of the invention, the viral ultrafiltration step is carried out after the step of filtering the clean crude extract (C) and still preferably after the sub-step of removing impurities from the clean crude extract. The viral ultrafiltration step is preferably carried out on the purified extract.

Such a succession of steps is particularly advantageous for the purpose of solving problems known in the state of the art, such as packing of the filters and variability of the viral ultrafiltration process.

Note that the purified allergenic extract subjected to the viral ultrafiltration step, and thus substantially free of viruses and/or mycoplasmas, is preferably characterized by a protein dosage ranging from 100% to 300% by weight of the expected protein dosage.

Note that the purified allergenic extract subjected to the viral ultrafiltration step, and thus substantially free of viruses and/or mycoplasmas, is preferably characterized by a microbial load <100,000 CFU/ml, preferably ≤50,000 CFU/ml.

### 3rd Embodiment

According to a third preferred embodiment of the production process, the viral ultrafiltration step is carried out before packaging.

Still preferably, the viral ultrafiltration step is carried out on the finished product, to be understood as the product obtained from the step of microbial filtration of the purified and titrated extract (E).

In accordance with this second embodiment, the finished product to be subjected to viral ultrafiltration may contain one or more of the allergens among those usable for the purposes of the present invention.

Note that, optionally, the aforesaid first, second and third embodiments may be simultaneously implemented in a same process for producing a finished product containing or consisting of a purified extract of an allergen.

Allergens and Products that Contain them.

For the purposes of the present invention, the allergen is preferably selected from
mite allergens, the mites preferably being Dermatophagoides farinae or Dermatophagoides pteronyssinus or Euroglyphus maynei;
hymenoptera venom allergens, the hymenoptera being preferably selected from Apis mellifera, Vespa Crabro, Vespa velutina, Vespa mandarinia, Vespa velutina, Vespula germanica, Vespula vulgaris, Polistes dominula, Polistes exclamans, Bombus terrestris, Bombus agrorum, Bombus medics, Dolichovespula, Vespula alascensis, Vespula maculifrons, Vespula flavopilosa, Vespula pensylvanica and Vespula Squamosa, still preferably selected from Apis mellifera, Vespa Crabro, Vespula germanica, Polistes exclamans, Bombus terrestris, Bombus agrorum, Bombus medics, Polybia paulista;
pet animal allergens, pet animals being preferably Canis lupus or Felis catus.

In the preferred embodiment, the allergens of interest for the embodiment of the invention are the mite allergens (Dermatophagoides farinae or Dermatophagoides pteronyssinus or Euroglyphus maynei) and allergens from cats (Felis catus).

The allergens of interest within the purposes of the invention are preferably characterized by molecular weight ranging from 1 kDa to 150 kDa.

Still preferably, the mite allergens have molecular weight ranging from 10 kDa to 150 kDa. Still preferably, the hymenoptera venom allergens have molecular weight ranging from 1 kDa to 150 kDa, preferably from 1 kDa to 100 kDa.

Still preferably, the per animal allergens have molecular weight ranging from 5 kDa to 150 kDa.

A further object of the present invention is a finished product for medical use or for in vivo diagnostic use or for in vitro diagnostic use, comprising one or more allergenic extracts, said product being substantially free of viruses and/or mycoplasmas.

Such finished product is preferably obtained with the process object of the claims.

According to a preferred embodiment, the finished product that can be obtained with the process of the present invention constitutes:
a pharmaceutical product for medical use, which can be used as such in the treatment or in the prevention of allergies;
a raw material usable for providing pharmaceutical compositions for medical use, in the treatment or in the prevention of allergies.

Preferably, the finished products in accordance with the present invention are intended for medical use in specific, preventive or therapeutic immunotherapy of allergic subjects. Still preferably, to the medical use in the treatment of clinical manifestations from allergy selected from the group consisting of: mild and/or moderate atopic dermatitis, rhinitis or rhino-conjunctivitis; mild and/or moderate asthma.

Still preferably, the pharmaceutical compositions in accordance with the present invention are intended for medical use in the prevention of allergic march.

It should be noted that, preferably, the finished products in accordance with the invention are usable both in the induction step of the specific immunotherapy treatment, and in the maintenance step.

In the induction step, the dose of allergen is gradually increased until the maintenance dose is reached.

Preferably, the pharmaceutical compositions in accordance with the present invention are in the form of a sublingual or injectable, preferably sublingual, composition.

In the case of injectable compositions, preferably subcutaneous, the purified allergen extract may be further manipulated and modified prior to inclusion in the finished pharmaceutical composition. In particular, in order to make it suitable for the route of administration for which it is intended, the allergen can be adsorbed on L-tyrosine crystals, according to techniques known in the state of the art; it can be subjected to further microbial, viral and/or mycoplasmic filtration processes, in order to make the finished product substantially sterile and suitable for subcutaneous administration.

These further filtration steps may require working in sterile environments of higher classification than those of grade C according to ISO 14644-1 such as grade B and/or A environments according to ISO 14644-1.

Alternatively, the finished products in accordance with the present invention are intended for use in the in vivo or in vitro diagnosis of allergies, preferably in the in vivo or in vitro diagnosis of respiratory or contact allergies.

According to a preferred embodiment, the finished product that can be obtained with the process of the present invention constitutes:
a pharmaceutical product for in vivo or in vitro diagnostic use, usable as such in the detection of allergies;
a raw material usable for providing pharmaceutical compositions for in vivo or in vitro diagnostic use in the detection of allergies.

Examples of applications in vivo of the finished product for diagnostic use are
the patch test, i.e. a diagnostic test useful for the identification of contact allergies (DAC) consisting of applying suspicious substances (haptens) on the back of the subject to be tested, these substances being placed in cells fixed with patches. The patches are generally kept for 48 hours on site before removal and evaluation of the appearance of any erythema, oedema and vesicles that could define allergen positivity.
the prick test, i.e. a skin test used to diagnose allergies, preferably respiratory allergies, which consists in applying a predetermined concentration of allergen to the skin and in lightly pricking the skin with a special lance to facilitate the penetration of the substance into the superficial layers of the skin. Alternatively, it is possible to carry out an “intradermal test” that envisages using a larger needle, which favours the subcutaneous injection of the allergen.

The finished product in accordance with the invention may also be used in carrying out in vitro diagnostic tests, which may accompany or replace the use of in vivo diagnostic tests. Examples of applications in vitro of the finished product for diagnostic use are
specific IgE dosage, using for example ELISA assays
dosing of molecules linked to the activation of basophils (histamine, heparin, neutral proteases, acid hydrolases and other chemotactic factors), using for example CAST-ELISA assays (which measure the sulphide leukotrienes produced in vitro after stimulation of the basophils with an allergen).

Preferably the finished products of the invention, regardless of whether they are intended for medical or diagnostic use, may comprise one or more of the aforementioned allergens.

According to a preferred embodiment, finished products, substantially free of viruses and/or mycoplasmas, in accordance with the present invention, comprise purified allergen extracts from Dermatophagoides farinae or Dermatophagoides pteronyssinus or Euroglyphus maynei. Still preferably, the pharmaceutical composition comprises the aforesaid extracts from mites in 1:1:1 weight ratio.

According to an alternative embodiment, finished products in accordance with the present invention comprise purified extracts, substantially free of viruses and/or mycoplasmas, of allergens from hair and dandruff from cats (Felis catus).

### Examples

For illustrative and non-limiting purposes, the description of the process of production of the purified extract, substantially free of viruses and/or mycoplasmas, in accordance with the present invention is reported below.

The following example refers to the process for manufacturing a purified extract of Euroglyphus maynei, but applies as such also in the case of manufacturing purified extracts of D. pteronyssinus, D. farinae.

### 1. Process for Manufacturing a Purified Extract of Euroglyphus maynei

### Glossary

SM: Source Material, raw material
EM1 API: active substance, standardized Mother extract in total proteins having its own formulation.
Extraction ratio (w/V): ratio between quantity of Source Material weighed in the weighing room (grams) and volume of phosphate buffer solution pH 8.0 (mL) necessary to perform the step of the cold extraction process.

### Introduction

The set-up of the EM1 API described in detail below is made within and no later than 42 hours from the start of processing (collection of the Source Material from the freezer), the set-up time of the active substance verified during the process validation. There are no time interruptions during the manufacturing process.

All the processings are carried out at a controlled temperature (room temperature: 18÷22° C.), except for the steps of extraction and centrifugation of the suspension by separation of the steps that are carried out at lower temperatures.

Table 2 shows the composition of the EM1 API of Euroglyphus maynei:

TABLE 2

| | |
|---|---|
| Name: | Mother extract of Euroglyphus maynei |
| Components: | Allergenic protein aqueous solution of |
| | Euroglyphus maynei 2.0 mg/mL (Lowry) |
| | Water for injectable preparations q.s. |
| | Sodium chloride EP 9 mg/mL |

The formulation of the Extraction of the EM1 API of Euroglyphus maynei is shown in Table 3:

TABLE 3

| | |
|---|---|
| Extraction | 100 g (±0.5 g) / 1000 mL (w/V) |
| Formulation: | 100 g SM Culture of Euroglyphus maynei |
| | 1000 mL of phosphate buffer pH 8.0 |

Set-Up Principle of EM1 API of Euroglyphus maynei:

The manufacturing process envisages the following main process steps:
1) Collection of Source Material and solutions (Step 1A); transfer of the materials to the weighing room, thawing and weighing of the SM (Step 1B);
2) Cold extraction of the SM of Euroglyphus maynei in phosphate buffer solution pH 8.0;
3) Separation of the phases: Centrifugation extracted by separation of liquid phase from solid phase (supernatant and precipitate);
4) Pre-filtration and filtration: pre-filtration (8 μm) and filtration of the extract (0.45 μm+0.2 μm);
5) Purification of the extract (nominal cut off 5000 Dalton): Diafiltration and subsequent filtration of the diafiltered EM1 (0.2 μm+0.1 μm or 0.2 μm);
6) Ultra-Filtration (0.1 μm pre-filter+20 nm ultra-filter);
7) Return to protein titre: sampling of the diafiltered and ultrafiltered extract (untitrated EM1) for carrying out in the CQ laboratory the protein dosage, on the basis of which the back titration is carried out (specification of Lowry protein dosage: 1.9-2.4 mg/mL) with saline solution;
8) Filtration of the titrated EM1 (0.2 μm) and filling of the containers: Sampling of the titrated EM1 for carrying out the microbial load and protein dosage, and subsequent filtration at 0.2 μm, with simultaneous filling of the sterile containers; sampling for release analysis.

9) Transfer and storage in the dedicated Quarantine area.

### Description of the Manufacturing Process

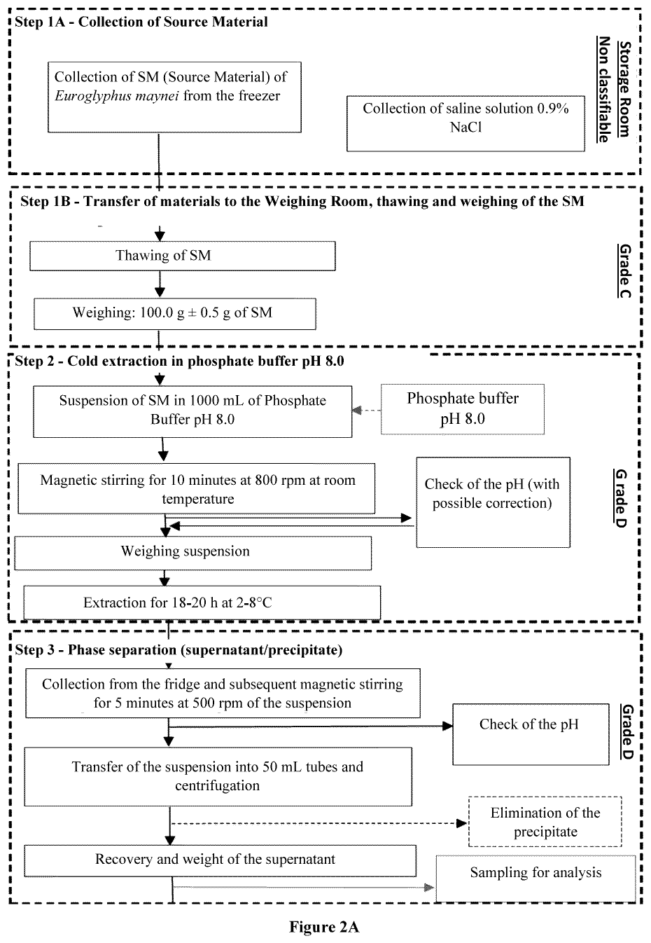

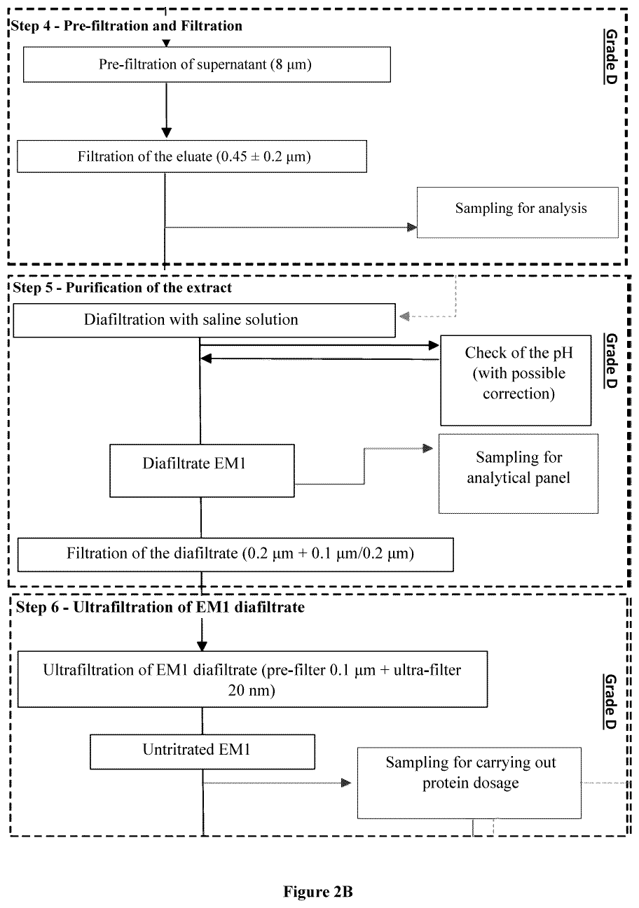

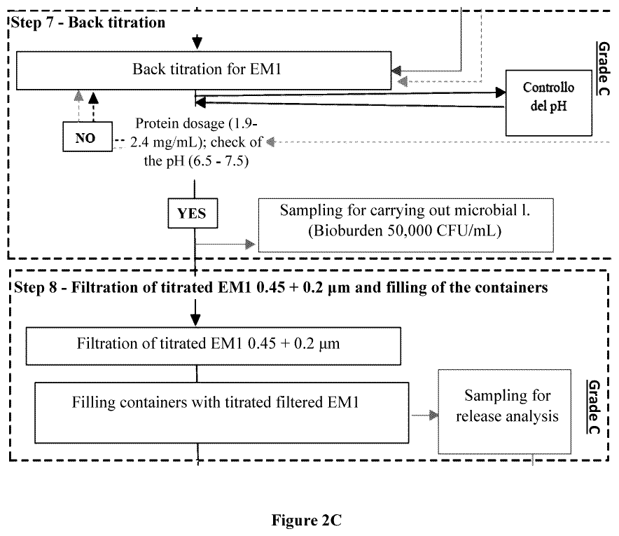

The flowchart of the process for manufacturing the EMI API of Euroglyphus maynei is the one depicted in FIG. 2.

Step 1-Collection and Thawing of the Source Material and Transfer of the Materials from the Storage to the Weighing Room (Grade C Room).
1) Take the SM aliquot from the freezer.
2) Allow the SM to thaw before weighing.
3) Under hood, weigh the desired aliquot of SM on technical scales in the dedicated plastic containers and seal the container.
4) Transfer the materials to the grade D Room dedicated to extraction.

### Step 2-Cold Extraction in Phosphate Buffer pH 8.0 in the Extraction/Modification Room (Grade D Room).

1) Take the bottle containing the phosphate buffer pH 8.0 from the fridge and place all the materials under a chemical hood.
2) Place the SM in a sterile bottle with stir bar at the bottom, add phosphate buffer pH 8.0, close the bottle and stir manually to allow to fully wet the SM.
3) Transfer the remaining volume of the phosphate buffer pH 8.0 into the extraction bottle and stir.
4) Place the extraction bottle on the stirrer under constant stirring in order to favour the mixing of the phases. At the end of stirring, check that the pH has not changed and, if necessary, correct it.
5) Close the bottle and weigh it on technical scales.
6) Transfer the bottle to the fridge for the cold extraction step.

### Step 3-Separation of the Phases (Supernatant/Precipitate) in the Extraction/Modification Room (Grade D Room).

1) Check the cold extraction step;
2) Take the extraction bottle from the fridge;
3) Place the extraction bottle under stirring;
4) Under constant stirring, transfer the suspension to tubes for carrying out centrifugation.
5) At the end of centrifugation, transfer the supernatant (extract) into a sterile bottle.
6) Sample the supernatant for the control of protein dosage and carrying out the microbial load (Protein dosage: 3.0-9.0 mg/mL; Total microbial load before filtration: Bioburden lower than or equal to 1×10¹⁰ CFU/mL).

### Step 4-Pre-Filtration and Filtration in the Extraction/Modification Room (Grade D).

1) Pre-Filtration of the extract by PP filter with 8 μm porosity:
   Place the bottle used to collect the extract to be pre-filtered closed with a screw cap on the scales and weigh it;
   Open the bottle intended for collecting the filtrate and mount the filter system with a filter with a porosity of 8 μm. The system consists of a silicone tube (priming) with the filter mounted at one end housed on the bottle of the filtrate, with direction “in”→“out” from the bottle of the supernatant to be filtered up to the bottle of the filtered supernatant.
   Operate the peristaltic pump and wait for the entire volume to be filtered.
2) Filtration of the extract with PES filter with porosity 0.45 μm and 0.2 μm:
   Open the bottle intended for collecting the filtrate and mount the filter system with a filter with a porosity of 0.45 μm and 0.2 μm. The system consists of a silicone tube (priming) with the filter mounted at one end housed on the bottle of the filtrate, with direction “in”→“out” from the bottle of the pre-filtrate to be filtered up to the bottle of the filtrate.
   Operate the peristaltic pump and wait for the entire volume to be filtered.
   Take a sample to carry out the analytical panel (Protein dosage: 3.0-9.0 mg/mL; Total microbial load before purification: Bioburden lower than or equal to 50,000 CFU/mL; protein profile: the trace must contain the bands 26.4 kDa; 18.0 kDa).

### Step 5-Purification of the Extract in the Diafiltration/Dialysis/Ultrafiltration Room (Grade D)

1) Purification with diafiltration technique with volume reduction: the filtered extract is diafiltered using a diafiltration membrane with nominal cut off of 5,000 Daltons. The latter consists of a series of selective membranes with variable porosities, which have the task of completely removing or lowering the concentration of salts, proteins and molecules with molecular weight of less than 5,000 Daltons, which pass through the membrane and are not retained (this volume of waste is called permeate or filtrate). The remaining part of the extract in diafiltration, retained by the membrane and containing proteins and molecules with a molecular weight higher than 5,000 Daltons is called retentate.

The diafiltration with volume reduction envisages the following process steps (one passage of concentration of the extract and four passages of washing with saline solution, V1-V4). At each passage, a sample is taken for carrying out an analytical panel to evaluate that the diafiltration protein profile is compliant with respect to a standard profile (Protein dosage of each diafiltration step: retentate protein dosage V1: 3.0-9.0 mg/mL; V2: 2.5-7.5 mg/mL; V3-V4: 2.0-6.0 mg/mL):
Each diafiltration passage is performed at a constant pressure of 2 bar until all the extract is inside the system. The extract passes through the diafiltration membrane and is purified from salts, proteins and molecules with a molecular weight lower than 5,000 Daltons, undergoing a concentration.
Turn off the peristaltic pump. Open the valve of the retentate and converge the entire volume inside a cylinder.
Check that the pH has not changed and correct it if necessary.
With the aid of sterile pipettes of adequate volume, take a sample of the EM1 to perform the analytical panel (protein dosage: 2.0-6.0 mg/mL; protein profile: the trace must contain the bands 26.4 kDa, 18.0 kDa; Microbial load before ultrafiltration: Bioburden lower than or equal to 50,000 CFU/mL).
   Filtration of the Extract with PES Filter with 0.2 μm Porosity or with 0.2 μm and 0.1 μm Porosity:
Open the bottle intended for collecting the filtrate and mount the filter system with a filter with 0.2 μm porosity or 0.2 μm and 0.1 μm porosity. The system consists of a silicone tube (priming) with the filter mounted at one end housed on the bottle of the filtrate, with direction “in”-> “out” from the bottle of the purified matter to be filtered up to the bottle of the filtrate.
Operate the peristaltic pump and wait for the entire volume to be filtered.
2) The subsequent step takes place in the same room.

### Step 6-Ultrafiltration of the Diafiltrate EMI in the Diafiltration/Dialysis Room (Grade D).

The ultrafiltration step uses two filters in a single process step: pre-filter with 0.1 μm porosity connected in series to the ultrafilter, with viral retention capacity with 20 nm porosity. The ultrafilter is connected to a Vent filter (0.2 μm).

Filtration takes place under constant pressure of 2 bar:
1) The system consists of a steel container in which the extract to be ultrafiltered is placed equipped with the following connections:
   “In”-connection: inlet to the filtered oil-free compressed air container at 0.2 μm at the point of use at a constant pressure of 2 bar.
   “Out”-connection: connection of the container to pre-filter and ultra-filter. The ultrafilter has two outlets connected in turn to two silicone tubes housed in the ultrafiltered extract collection cylinder.
2) Perform the following operations:
   Condition the filters with saline solution;
   Place the extract to be ultrafiltered in the container;
   Bring the pressure to 2 constant bars and wait for the complete passage of the volume in the collection cylinder.
3) With the aid of sterile pipettes of adequate volume, take a sample of the EMI to perform protein dosage (Protein dosage: 2.0-6.0 mg/mL).
4) Transfer the untitrated EMI to the grade C room dedicated to Non-Sterile Preparation for the subsequent step.

### Step 7-Back Titration of the Untitrated EMI in the Non-Sterile Preparation Room (Grade C).

1) Place on the work surface the bottle containing the saline solution for back titration and the untitrated EM1.
2) Based on the result of the protein dosage (Step 6.3), place the untitrated EM1 under constant magnetic stirring at room temperature and then back titrate with saline solution (titre EM1 titrated: 1.9-2.4 mg/mL).
3) Continue the magnetic stirring.
4) Take a sample for carrying out protein dosage before the filtration step (Protein dosage: 1.9-2.4 mg/mL).
5) Before moving on to the subsequent step, with the aid of sterile pipettes sample the titrated EMI for carrying out the microbial load before filtration (Total microbial load before filtration: Bioburden lower than or equal to 50,000 CFU/mL).

### Step 8-Filtration 0.45+0.2 μm Titrated EM1 in the Non-Sterile Preparation Room (Grade C).

1) Based on the weight of the titrated EM1 obtained, set up a number of bottles for the collection of the filtrate.
2) On the LAF work surface, for each container, perform the following operations:
   Place the bottle closed with screw cap on the scales and press the “tare” key;
   Open the bottle intended for collecting the filtrate and mount the filter system with PES filter with 0.45+0.2 μm porosity. The system consists of a silicone tube (priming) with the filter mounted at one end housed on the bottle of the filtrate, with direction “in”→“out” from the bottle of the titrated EM1 to be filtered up to the bottle of the filtrate.
   Operate the peristaltic pump and wait for the desired volume to be filtered.

### 2. Analysis of the Bacterial Endotoxin Content after Nanofiltration

### Glossary

LAL: Lysate of amoebocytes of Limulus polyphemus. These amoebocytes have the characteristic of reacting with endotoxins and of causing in vivo the coagulation of the blood of Limulus. This property is exploited in the test for endotoxin determination.
Limulus polyphemus: an ancient arachnid member of the Merostom class which lives only on the Atlantic coast of the United States.

Lipopolysaccharides (LPS): pyrogenic components of the outer cell membrane of Gram-bacteria, consisting of a lipid and a polysaccharide portion, capable of triggering the immune response in animals.
EM1: Mother extract
EM1 API: active substance, standardized Mother extract in total proteins having its own formulation.
R%=Recovery expressed as a percentage (range: 50-200%). Expresses the degree of significance of the test. The Recovery % range is set according to what is reported in the current Ph.Eur. ed. monograph of reference.

### Introduction

The bacterial endotoxins are lipopolysaccharides (LPS), cell wall components of Gram-negative bacteria that can cause fever (pyrogens) when injected into the bloodstream. They are constantly released into the environment where the live bacteria are present and totally released following the death of the bacteria themselves. Although there is a small number of pyrogens with structure other than LPS, it can reasonably be said that the absence of bacterial endotoxins in a product implies the absence of pyrogenic components.

Bacterial endotoxins resist high temperatures (up to 200-250° C.), pass through the sterilising filters commonly used for the pharmaceutical preparations, are water-soluble and non-volatile so it is difficult, if not impossible, to remove them from an injectable preparation.

### Method

The kinetic-chromogenic LAL method is an analytical method for determining the presence and for quantifying the bacterial endotoxins in a sample. The principle on which this method is based consists in measuring the reaction time necessary for the development of a colour intensity, following the reaction that occurs between LAL and the bacterial endotoxins.

For the purpose of the test, the Endosafe® Nexgen-PTS™ system has been used, which is based on the measurement of the colour intensity, a parameter directly linked to the concentration of endotoxin in the sample. The colour intensity formed by the release of the chromophore is measured by a spectrophotometer set at an appropriate wavelength. The chromophore, which causes the appearance of a yellow colour, is measured photometrically at 385-410 nm by the reader.

The time required for the appearance of the colour depends on the amount of endotoxin present: in the presence of large amounts of endotoxin the reaction takes place quickly; on the contrary, if the amount of endotoxin is low, the reaction takes place slowly.

The cartridge system of the Endosafe-PTS instrument contains a precise amount of LAL (FDA-authorized), a chromogenic substrate and Standard Endotoxin Control (CSE). Each cartridge has four channels to which the LAL reagent and the chromogenic substrate have been added. In addition, the second and fourth channels also contain endotoxin spike for the positive control.

### Results

TABLE 4
The use of the Viral ultrafiltration step (Step 6) allowed a reduction by
about 2.38 Log₁₀ (or by about 99.58%) of the endotoxin content compared
to what was detected in the sample of the crude extract (Phase 3).
EM1 API Euroglyphus maynei

| | Dilution | | | | | Total |
|---|---|---|---|---|---|---|
| Process step | used | Result | | R % | Outcome | proteins |
| Step 3 - Phase separation | 1:100000 | 154888 | EU/mL | 117% | Valid | 6.1 mg/mL |
| (supernatant/precipitate) | | | | | | |
| Step 4 - | 1:100000 | 191161 | EU/mL | 146% | Valid | 6.0 mg/mL |
| Prefiltration/Filtration | | | | | | |
| Step 5 - Purification of | 1:100000 | 226464 | EU/mL | 128% | Valid | 4.7 mg/mL |
| the extract | | | | | | |
| Step 6 - Ultrafiltration | 1:100000 | <5000 | EU/mL | 104% | Valid | 3.8 mg/mL |
| | 1:10000 | <500 | EU/mL | 93% | Valid | |
| | 1:1000 | 651 | EU/mL | 134% | Valid | |
| Step 8 - Filtration of | 1:1000 | 144 | EU/mL | 99% | Valid | 1.9 mg/mL |
| titrated EM1 and filling | | | | | | |
| of the containers (release | | | | | | |
| analysis) | | | | | | |

TABLE 5
The use of the Viral ultrafiltration step (Step 6) allowed a reduction by
about 2.47 Log₁₀ (or by about 99.66%) of the endotoxin content compared to
what was detected in the sample of the crude extract (Phase 3).
EM1 API Dermatophagoides pteronyssinus

| | Dilution | | | | | Total |
|---|---|---|---|---|---|---|
| Process step | used | Result | | R % | Outcome | proteins |
| Step 3 - Phase separation | 1:100000 | <5000 | EU/mL | 92% | Valid | 5.1 mg/mL |
| (supernatant/precipitate) | 1:10000 | 1208 | EU/mL | 132% | Valid | |
| Step 4 - | 1:10000 | 888 | EU/mL | 86% | Valid | 4.9 mg/mL |
| Prefiltration/Filtration | | | | | | |
| Step 5 - Purification of | 1:10000 | 966 | EU/mL | 102% | Valid | 3.7 mg/mL |
| the extract | | | | | | |
| Step 6 - Ultrafiltration | 1:10000 | <500 | EU/mL | 78% | Valid | 3.1 mg/mL |
| | 1:1000 | <50 | EU/mL | 95% | Valid | |
| | 1:100 | 17.1 | EU/mL | 159% | Valid | |
| Step 8 - Filtration of | 1:100 | <7.24 | EU/mL | 138% | Valid | 2.3 mg/mL |
| titrated EM1 and filling | | | | | | |
| of the containers (release | | | | | | |
| analysis) | | | | | | |

1. A process for producing a finished product containing or consisting of a purified extract of an allergen, the process comprising the steps of:
A) providing a crude extract of the allergen, in which the allergen is of natural origin and is obtainable by isolation from a biological sample;
B) optionally, cleaning the crude extract, to obtain a clean crude extract, the step of cleaning comprising at least the sub-step of
B1) filtering the crude extract to remove the microbial load of the biological sample;
C) optionally, purifying the crude extract or the clean crude extract, to obtain a purified extract, the step of purifying comprising at least the sub-step of
C1) filtering the crude extract or the clean crude extract to remove impurities having a molecular weight ≤5,000 Da,
the process wherein one or more viral ultrafiltration steps.

2. The process according to claim 1, wherein the one or more viral ultrafiltration steps are carried out in a filter assembly comprising:
a filter with a porosity of dimensions ranging from about 20 nm to 100 nm;
preferably, a pre-filter, coupled at the up front of the filter, with a porosity of dimensions ranging from about 100 nm to 900 nm.

3. The process according to claim 1, wherein the one or more viral ultrafiltration steps are carried out at a constant pressure ranging from 0.5 bar to 5 bar.

4. The process according to claim 3, wherein the one or more viral ultrafiltration steps are carried out in a time interval ranging from 2 to 24 hours.

5. The process according to claim 1, wherein the sub-step of filtering the crude extract (B1) comprises the sub-steps of:
performing a first dimensional filtration, using a first filter with a porosity of dimensions ranging from 8 μm to 20 μm;
performing a second dimensional filtration, after the first, using a second filter with a porosity of dimensions ranging from 0.1 μm to 0.2 μm.

6. The process according to claim 1, wherein the sub-step of filtering the clean crude extract (C1) is carried out by diafiltration, preferably by tangential diafiltration.

7. The process according to claim 1, wherein the clean crude extract is characterized by a microbial load <100,000 CFU/ml.

8. The process according to claim 1, wherein the allergen is selected from the group consisting of mite allergens, pet allergens, hymenoptera venom allergens.

9. The process according to claim 1, wherein the allergen has a molecular weight ranging from 1 kDa to 150 kDa.

10. The process according to claim 1, wherein at least the steps of providing the crude extract (A), cleaning the extract (B) and purifying the extract (C) are carried out in grade C and/or D sterile environments according to ISO 14644-1.

11. The process according to claim 1, wherein the entire purification process is carried out in or within 42 hours.

12. A finished product for medical use or finished product for in vivo diagnostic use comprising one or more allergen extracts, which is obtainable with the production process according to claim 1.

13. The finished product for use according to claim 12,
in specific, preventive or therapeutic immunotherapy of allergic subjects; or
in the in vivo diagnosis of allergies, preferably respiratory or contact allergies.

14. The finished product for medical use according to claim 12, in the form of a sublingual or injectable preparation.

15. Use of the finished product according to claim 12 for the in vitro diagnosis of allergies, preferably respiratory or contact allergies.
